(19) United States
(12) Patent Application Publication
(10) Pub. No.: US 2017/0137749 A1
(43) Pub. Date: May 18, 2017

(54) ADDITIVES AND COMPOUND DETERGENTS

(71) Applicants: Karen Marie McMurray, Mission Viejo, CA (US); Peter Mattei, Lemon Grove, CA (US)

(72) Inventors: Karen Marie McMurray, Mission Viejo, CA (US); Peter Mattei, Lemon Grove, CA (US)

(73) Assignee: True Blue Science, LLC, Mission Viejo, CA (US)

(21) Appl. No.: 15/336,771

(22) Filed: Oct. 27, 2016

Related U.S. Application Data
(60) Provisional application No. 62/255,196, filed on Nov. 13, 2015.

(51) Int. Cl. C11D 1/66; C11D 3/32; A61Q 19/10; C11D 17/00; A61K 8/42; A61K 8/34; C11D 11/00; C11D 17/06

(52) U.S. Cl. CPC C11D 1/66; C11D 11/0023; C11D 11/0017; C11D 3/32; C11D 17/06; C11D 17/0008; A61K 8/42; A61K 8/34; A61Q 19/10

(57) ABSTRACT

Compound detergents, comprising a low HLB surfactant noncovalently bonded with a high HLB surfactant are presented. The low HLB surfactant and high HLB surfactant are preferably present in the compound detergent in a ratio of between 2:1 and 1:10 by weight, inclusive. Such compound detergents are formulated to remain homogenously distributed in an aqueous medium for a period of at least one hour. The inventive compound detergents can clean polymeric textiles without the use of solvents and while the surfaces are continuously exposed to water or a water solution. Additives for detergents comprising high HLB surfactants configured to noncovalently bond with low HLB surfactants in the detergents are also presented.

### FIELD OF THE INVENTION

The field of the invention is compositions and methods of formulating additives and detergents for removal of hydrophobic stains from polymeric surfaces while continuously exposed to water, for example, where the polymeric surface is submerged in water or a water solution, or where the stain is removed by a towel or towelette including the detergent and water.

### BACKGROUND

The following background discussion includes information that may be useful in understanding the present invention. It is not an admission that any of the information provided herein is prior art or relevant to the presently claimed invention, or that any publication specifically or implicitly referenced is prior art.

There are many challenges in developing aqueous systems to clean sticky greases. To improve the performance of aqueous cleaners, solvents, surfactants, chelating agents, emulsifiers are added. However, solvents used to dissolve greases typically are poorly soluble in water. Organic solvents such as methylene chloride, glycol ethers, d-limonene, hydrocarbons (e.g., hexanes), kerosene, xylene, methanol, etc. are typically volatile or exhibit toxic effects, making them impractical for household applications. Additionally, such additives can be toxic and damage the environment, sometimes requiring separate waste collection and disposal.

One solution is to use caustic, high pH (pH>10) cleansers. However, these cleaners can cause burns, so users must wear protective gloves and clothing. Caustic cleansers can also damage textiles and delicate fabrics making them impractical for use as laundry detergents.

Monounsaturated fatty amides are attractive alternatives to solvent-based stain removers for the removal of hydrophobic stains from many substrates. For example, N,N-dimethyl 9-decenamide (marketed by Stepan Company under the name STEPOSOL® MET-10U (“MET-10U”)), MET-10U is a low hydrophile-lipophile balance (“HLB”) surfactant that can replace solvents such as methylene chloride, glycol ethers, d-limonene, hydrocarbons (e.g., hexanes), kerosene, xylene, methanol, etc. In hard surface-cleaning applications, MET-10U has been shown to efficiently clean metals and remove graffiti, adhesives, and baked on soils. Additionally, MET-10U effectively removed dried-on paint from paint brushes. Anhydrous MET-10U can be loaded onto carriers such as zeolites, metal-organic frameworks, and nano- and microcellulose.

Unfortunately, MET-10U alone does a poor job of removing sticky greases from polymeric substrates, because the monounsaturated tail sticks to the substrate. Typically, solvents must be used to wash away the MET-10U and the stain, because MET-10U is poorly soluble in water (typically, 0.2-0.8%). Thus, MET-10U is not truly a solvent alternative for the cleaning of polymeric substrates. Further, the low HLB MET-10U would apparently not be suitable for stain removal from a surface that is continuously exposed to water during the removal process.

Masters et al. disclose aqueous hard surface cleaners based on monounsaturated fatty amides in U.S. Patent Pub. No. 2015/0225674 (filed Mar. 5, 2015). Specifically, Masters teaches hard surface cleaner concentrates that include a monounsaturated N, N-dialkylamide and a surfactant. Masters's hard cleaner concentrates have a pH within the range of 6.0 to 9.0, and as such serve as alternatives to high pH (pH>10) caustic cleaners. However, Masters fails to appreciate that low HLB surfactants can be blended with high HLB surfactants (HLB≧13) to obtain compound detergents that clean oily soils from polymeric textiles while continuously exposed to water. Moreover, Masters fails to contemplate that anhydrous compound detergents can be further compounded with effervescent carriers.

This and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

### SUMMARY OF THE INVENTION

It has yet to be appreciated that a surfactant having a high HLB (e.g., greater than 13 HLB) in combination with MET-10U in aqueous formulations can be used to remove hydrophobic stains from polymeric substrates and even skin, especially where the substrate is continuously exposed to water during the stain removing process.

The inventive subject matter provides compositions and methods of using a compound detergent. Compound detergents according to the inventive subject matter comprise a low HLB surfactant noncovalently bonded with a high HLB surfactant to form the compound detergent. The low HLB surfactant and high HLB surfactant are preferably present in the compound detergent in a ratio of between 2:1 and 1:10 by weight, inclusive. For example, the ratio of low HLB surfactant to high HLB surfactant can be approximately 2:1,1:1, 1:2, 1:3, 1:4, 1:5, 1:6, 1:7, 1:8, 1:9 or 1:10. As used herein, the term approximately means within 10%. For example, “approximately 2:1” includes a range between 1.8:1-2.2: 1, inclusive. “Approximately 1:5” includes a range between 1:4.5-1:5.5, inclusive.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

In some embodiments of the inventive subject matter, the compound detergent is formulated to remain homogenously distributed in an aqueous medium for a period of at least one hour (e.g., at least 5 hours, at least 10 hours, at least 24 hours, at least 48 hours). As used herein, the term “homogenously distributed” means substantially the same amount of compound detergent (i.e., within 10%) is present in any unit volume of an aqueous medium. This extended homogenous distribution can allow the additives and detergents of the inventive subject matter to be used to remove stains from substrates while the substrates are continuously exposed to water or a water solution (e.g., when the substrate is submerged at least partially in water, when soils are removed using a towel impregnated with the detergent and water, when soils are removed with a towellete including water and the compound detergent).

Contemplated detergents can be solutions or colloidal suspensions. In solutions, the low HLB surfactant, high HLB surfactant, and aqueous media are noncovalently bonded (e.g., by van der Waals forces, hydrophobic interactions, polar aprotic interactions, and hydrogen bonds), forming a homogeneous mixture. The high and low HLB surfactants may also form colloidal suspensions (emulsions) of micelles/liposomes in water, wherein the high HLB surfactant stabilizes the micelle/liposome structures. Liposomal structures can have bi- or multi-layer structures and need not be spherical (contemplated liposomes can include ellipsoid, cylindrical, lamellar, and mixed structures).

In preferred embodiments, the low HLB surfactant comprises a monounsaturated fatty amide. In even more preferred embodiments, the monounsaturated fatty amide comprises N,N-dimethyl 9-decenamide.

With respect to the high HLB surfactant, the inventors contemplate that any surfactant having a HLB numbers greater than 10, more preferably between 13 and 20, inclusive, and even more preferably between 13 and 15, inclusive. Anionic, cationic, zwitterionic (amphoteric), and nonionic surfactants are suitable for use in the inventive compound detergents. Preferred high HLB surfactants include linear alcohol ethoxylates.

Therefore, the compound detergent can be formulated as liquid concentrates, dried anhydrous particles, or capsules. For example, contemplated formulations include powders, granules, pills, and tablets (coated and uncoated).

In yet further aspects of the inventive subject matter, compound detergents can be non-aqueous and compounded into an effervescing substrate. For example, the anhydrous compound detergent can be loaded onto the expanded substrates taught in International Patent Pub. No. WO2002036085 (filed Nov. 2, 2001) to Kayden. Kayden discloses effervescent compounds including an effervescent system comprising, for example, an expanded perborate salt, anhydrous, and oxoborate or an acid and one or more of sodium bicarbonate, sodium carbonate, potassium bicarbonate, and potassium carbonate, which are described as suitable carriers for surfactants, bleaching compositions, antiredeposition agents, binders, lubricants, colors, fragrances, corrosion inhibitors, disinfectants, pesticides, fertilizers and optical brighteners.

Advantageously, the inventive compound detergents are effective to remove oily soils from both hard surfaces as well as polymeric textiles, carpets, the human body, and dishware without the use of a solvent, and without damaging the washed surface. In an exemplary method of removing oily soils from a polymeric textile includes the steps of: (1) blending a high HLB surfactant with an anhydrous low HLB surfactant to form a water soluble concentrate wherein the high HLB surfactant is noncovalently bonded to the low HLB surfactant; and (2) using the concentrate in an aqueous medium in an amount effective to remove the oil soils from the polymeric textile while submerged or otherwise continuously exposed to water or a water solution.

In other aspects of the inventive subject matter, a high HLB surfactant can be used to extend the usefulness of a low HLB surfactant in a cleaning formulation for an aqueous solution. The high HLB surfactant is added to the cleaning formulation in an amount effective to homogenously distribute the low HLB surfactant in the aqueous solution without use of a solvent. Additionally, the low HLB detergent and the high HLB detergent are present in the aqueous solution in respective amounts sufficient to remove oily soils from a textile.

Various objects, features, aspects and advantages of the inventive subject matter will become more apparent from the following detailed description of preferred embodiments, along with the accompanying drawing figures in which like numerals represent like components.

### DETAILED DESCRIPTION

Surprisingly, the inventors discovered that addition of a high HLB surfactant to a low HLB surfactant yields a compound detergent that exhibits superior removal of hydrophobic stains from polymeric textiles, carpet, the human body, and dishware. In one example, a compound detergent removed 24% more of a standardized soil than commercial textile cleaning products. Compound detergents consistent with the inventive subject matter can readily be distributed homogenous in aqueous solutions, whereas low HLB surfactants alone are immiscible with water and stick to polymeric textiles. Because low HLB surfactants alone stick to polymeric textiles, they act as soils, which must be washed away with either solvents or another detergent. Advantageously, use of concentrates or aqueous solutions of such compound detergents in textile-cleaning applications eliminates such soiling without the use of solvents or a second washing step.

Exemplary compound detergents comprise a low HLB surfactant noncovalently bonded with a high HLB surfactant. With respect to the low HLB surfactant, suitable surfactants have an HLB number of less than 10. The inventors contemplate that various low HLB surfactants are suitable for use in the inventive compound detergents (e.g., 2,4,7,9-Tetramethyl-5-decyne-4,7-diol; BRIJ® 52 average Mₙ ˜330; BRIJ® 93 average Mₙ ˜357; BRIJ® L4 average Mₙ ˜362; Ethylenediamine tetrakis(ethoxylate-block-propoxylate) tetrol average Mₙ ˜7,200; IGEPAL® CA-520 average Mₙ ˜427; IGEPAL® CO-520 average Mₙ ˜441; MERPOL® A surfactant; MERPOL® SE surfactant; Poly(ethylene glycol) sorbitol hexaoleate; Poly(ethylene glycol)-block-poly(propylene glycol)-block-poly(ethylene glycol) average Mₙ ˜1,100; Poly(ethylene glycol)-block-poly(propylene glycol)-block-poly(ethylene glycol) average Mₙ ˜2,000; Poly(ethylene glycol)-block-poly(propylene glycol)-block-poly(ethylene glycol) average Mₙ ˜2,800; Poly(ethylene glycol)-block-poly(propylene glycol)-block-poly(ethylene glycol) average Mₙ ˜4,400; Poly(ethylene glycol)-block-poly(propylene glycol)-block-poly(ethylene glycol) average Mₙ ˜5,800; Poly(propylene glycol)-block-poly(ethylene glycol)-block-poly(propylene glycol) average Mₙ ˜3,300; Polyethylene-block-poly(ethylene glycol) average Mₙ˜1,400; Polyethylene-block-poly(ethylene glycol) average Mₙ ˜575; Polyethylene-block-poly(ethylene glycol) average Mₙ ˜875; Polyethylene-block-poly(ethylene glycol) average Mₙ ˜920; Sorbitan monopalmitate).

Especially suitable low HLB surfactants include monounsaturated fatty amides. Contemplated monounsaturated N,N-dialykyl amides include C₈-C₁₄ fatty amides. The alkyl moiety may comprise ethyl, propyl, and/or isopropyl groups, preferably methyl groups. For example N,N-dimethyl 7-octenamide; N,N-dimethyl 9-dodecenamide; N,N-dimethyl 9-tetradecenamide; N,N-diethyl 7-octenamide; N,N-diethyl 9-decenamide; N,N-diethyl 9-dodecenamide; N,N-diethyl 9-tetradecenamide; and most preferably N,N-dimethyl 9-decenamide (MET-10U).

In regard to suitable high HLB surfactants, anionic, cationic, zwitterionic (amphoteric), and nonionic surfactants that have HLB numbers between 10 and 20, inclusive, preferably between 13 and 15, inclusive are contemplated. Suitable anionic surfactants include amine oxides, alkyl sulfates, alkyl ether sulfates, olefin sulfonates, α-sulfonated alkyl esters (e.g., α-sulfonated methyl esters), α-sulfonated alkyl carboxylates, alkyl aryl sulfonates, sulfoacetates, sulfosuccinates, alkane sulfonates, alkylphenol alkoxylate sulfates, alkyl ether/ethoxy carboxylates, and the like, and mixtures thereof.

Additional suitable anionic surfactants have been described in McCutcheon's Detergents & Emulsifiers (M.C. Publishing, N. American Ed., 1993); Schwartz et al., Surface Active Agents, Their Chemistry and Technology (New York: Interscience, 1949); and in U.S. Pat. Nos. 4,285,841 and 3,919,678, which are incorporated herein by reference.

Contemplated cationic surfactants include fatty amine salts (including diamine or polyamine salts), quaternary ammonium salts, salts of fatty amine ethoxylates, quatemized fatty amine ethoxylates, and mixtures thereof. Further contemplated cationic surfactants are described in McCutcheon's Detergents & Emulsifiers (M.C. Publishing, N. American Ed., 1993); Schwartz et al., Surface Active Agents, Their Chemistry and Technology (New York: Interscience, 1949); and in U.S. Pat. Nos. 3,155,591; 3,929,678; 3,959,461; 4,275,055; and 4,387,090, which are incorporated herein by reference.

In one exemplary embodiment, a zwitterionic surfactant may be added to the low HLB surfactant to yield the inventive compound detergents. Suitable zwitter ionic surfactants include amine oxides, betaines, amphoacetates, amphopropionates, alkyl glycinates, and sulfobetaines. Specific examples include cocoamido-propylamine oxide, cetamine oxide, lauramine oxide, myristylamine oxide, stearamine oxide, alkyl betaines, cocobetaines, and amidopropyl betaines, (e.g., lauryl betaines, cocoamidopropyl betaines, lauramidopropyl betaines), and combinations thereof. Other suitable surfactants (amphoteric, anionic, cationic, and nonionic) are disclosed in U.S. Patent Pub. No. 2015/0225674, which is incorporated by reference.

In preferred embodiments of the inventive subject matter, the high HLB surfactant comprises a linear alcohol ethoxylate (e.g., TERGITOL™ linear alcohol ethoxylates and ECOSURF™ non-alkylphenol ethoxylates, both available from Dow Chemical Company). This discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Typically, the low HLB surfactant and high HLB surfactant are present in the compound detergent in a ratio of between 2:1 and 1:10 by weight, inclusive. The resulting compound detergents feature HLB balances between 2 and 10, preferably between 4 and 8, inclusive. The systems have typical Kauri-Butanol (KB) values of greater than 700, more typically greater than 800, and even more typically greater than 1000. These properties enable the inventive compound detergents to remove heavy hydrocarbons, paraffin, waxes, sebum, organic greases, and other soils. Advantageously, when used as laundry additive, the inventive compound detergents remove difficult soil burdens such as human waste and exudates (e.g., sebaceous secretions and sweat).

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Preferred compound detergents are formulated as a liquid concentrate, dried anhydrous particles, or a capsule. Another contemplated embodiment is a ready-to-use gel that exhibits shear-thinning under low-induced pressures. The concentrate is expected to exhibit no phase separation, evaporation, or degradation of the packaging, improving the shelf life of the detergent over solvent-based systems. Yet even more preferred compound detergents remain homogenously distributed in an aqueous medium for a period of at least 1 hour.

Therefore, it should be appreciated that concentrated anhydrous compound detergents can be produced by mixing the high and low HLB surfactants at ambient temperature and pressure for less than 20 minutes. Formulation can be performed at ambient temperature and pressure, because of the negligible volatility of the high and low HLB surfactants. The high and low HLB surfactants also blend efficiently, which saves in production energy costs. Typically, the blending speed is adjusted depending on the bulk weight inside the production mixers.

Solid formulations can be prepared by blending the high and low HLB surfactants with micro- and nano-cellulose. Cellulosic formulations are expected to aid nonredeposition and improve rheologic qualities. For preparation of solid/granular formulations, suitable dry mix blenders include “V” type, ribbon, or fluid bed mixers. For example, the mixing speed of a “V” blender can be adjusted to the bulk weight of each batch. The speed will preferably be high enough to agitate the carrier and additives, but slow enough for gravity fall dynamic mixing during each revolution. Under-blending can result in poor distribution and poor coverage across the surface area of the expanded carrier particles. Over-blending contributes to particle disruptions via friction and resulting loss of surface binding efficiency using the preferred carrier. For typical bulk densities and carriers, substantially homogenous products are typically obtained after 20 minutes of mixing time.

In yet further preferred embodiments, effervescing carrier systems can be employed for anhydrous compound detergents. Effervescent carrier systems include powders, pressed tablets, pills, and coated/uncoated granules. The inventors contemplate that any of the known effervescent systems can be used in conjunction with the inventive compound detergents. In one embodiment of the inventive subject matter, the compound detergent is combined with an effervescent composition (e.g., expanded perborate salt as described by Kayden (e.g., sodium perborate, anhydrous)) and the compound detergent can remain homogenously distributed while continuously exposed to water. This advantageously allows stains to be removed from substrates that are, for example, submerged in water. In other contemplated embodiments, a carbonate/acid-based effervescent system (e.g., sodium bicarbonate and citric acid).

The inventors contemplate that the technical effect of combining a high HLB surfactant with a low HLB surfactant is that the resulting compound detergent effectively removes oily soils from polymeric textiles, carpets, the human body, and dishware without the use of solvents.

In further aspects of the inventive subject matter, a method of removing oily soils from a polymeric textile comprises the steps of (1) blending a high HLB surfactant with an anhydrous low HLB surfactant to form a water soluble concentrate wherein the high HLB surfactant is noncovalently bonded to the low HLB surfactant and (2) using the concentrate in an aqueous medium in an amount effective to remove the oil soils from the polymeric textile. It should be appreciated that the considerations with respect to the low and high HLB surfactants discussed above also apply to the inventive methods.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A compound detergent, comprising:
a low HLB surfactant noncovalently bonded with a high HLB surfactant to form the compound detergent;
wherein low HLB surfactant and high HLB surfactant are present in the compound detergent in a ratio of between 2:1 and 1:10 by weight, inclusive; and
wherein the compound detergent is formulated to remain homogenously distributed in an aqueous medium for a period of at least 1 hour.

2. The detergent of claim 1, wherein the low HLB surfactant comprises a monounsaturated fatty amide.

3. The detergent of claim 2, wherein the monounsaturated fatty amide comprises N,N-dimethyl 9-decenamide.

4. The detergent of claim 1, wherein the high HLB surfactant comprises a linear alcohol ethoxylate.

5. The detergent of claim 1, wherein the high HLB surfactant exhibits an HLB of between 13-15, inclusive.

6. The detergent of claim 1, wherein the compound detergent is formulated as at least one of a liquid concentrate, dried anhydrous particles, and a capsule.

7. The detergent of claim 1, wherein the compound detergent is effective to remove oily soils from at least one of a polymeric textile, a carpet, a human body, and dishware without the use of a solvent.

8. A method of removing oily soils from a polymeric textile, comprising:
blending a high HLB surfactant with an anhydrous low HLB surfactant to form a water soluble concentrate wherein the high HLB surfactant is noncovalently bonded to the low HLB surfactant;
using the concentrate in an aqueous medium in an amount effective to remove the oily soils from the polymeric textile.

9. The method of claim 8, wherein the low HLB surfactant comprises a monounsaturated fatty amide.

10. The method of claim 9, wherein the monounsaturated fatty amide comprises N,N-dimethyl 9-decenamide.

11. The method of claim 8, wherein the high HLB surfactant comprises an alcohol ethoxylate.

12. The method of claim 8, wherein the low HLB surfactant and the high HLB surfactant are present in a ratio of between 2:1 and 1:10 by weight, inclusive.

13. The method of claim 8, further comprising a step of compounding the water soluble concentrate with an effervescent carrier.

14. The method of claim 8, wherein, during the step of using the concentrate to remove the oily soils from the polymeric textile, the polymeric textile is continuously exposed to water.

15. Use of a high HLB surfactant to extend the usefulness of a low HLB surfactant in a cleaning formulation for an aqueous solution, comprising:
adding the high HLB surfactant to the cleaning formulation in an amount effective to homogenously distribute the low HLB surfactant in the aqueous solution without use of a solvent; and
wherein the low HLB detergent and the high HLB detergent are present in the aqueous solution in respective amounts sufficient to remove oily soils from a textile.

16. The use of claim 15, wherein the low HLB surfactant comprises a monounsaturated fatty amide.

17. The use of claim 16, wherein the monounsaturated fatty amide comprises N,N-dimethyl 9-decenamide.

18. The use of claim 15, wherein the high HLB surfactant comprises a linear alcohol ethoxylate.

19. The use of claim 15, wherein the high HLB surfactant exhibits an HLB of between 13-15, inclusive.
